(19) United States
(12) Patent Application Publication
(10) Pub. No.: US 2026/0241193 A1
(43) Pub. Date: Aug. 20, 2026

(54) Alternating Polarity Magnetic Field Therapy Systems and Methods for Treatment of Medical Conditions

(71) Applicant: Asha Medical, Inc., San Ramon, CA (US)

(72) Inventor: Vivek K. SHARMA, San Ramon, CA (US)

(73) Assignee: Asha Medical, Inc., San Ramon, CA (US)

(21) Appl. No.: 19/540,754

(22) Filed: Feb. 15, 2026

Related U.S. Application Data
(60) Provisional application No. 63/759,530, filed on Feb. 17, 2025.

(51) Int. Cl. A61N 2/02

(52) U.S. Cl. CPC A61N 2/02

(57) ABSTRACT

Systems and method for destroying or inhibiting cancer cells and other dysfunctional cells include applying AP magnetic fields having a defined frequency of 5 Hz-500 kHz and a field strength of 0.1-5000 UT to a target body area that includes the dysfunctional cells, and modulating cellular stress responses in the cells. The magnetic field therapy (MFT) may be used to treat conditions including cancers, neurodegenerative diseases, metabolic disorders, and autoimmune disorders.

### CROSS-REFERENCE TO RELATED APPLICATION

This application claims the priority benefit of U.S. Provisional Application Ser. No. 63/759,530, filed Feb. 17, 2025 and entitled, “Alternating Polarity Magnetic Field Therapy Systems and Methods for Treatment of Medical Conditions,” which is hereby incorporated by reference in its entirety.”

### TECHNICAL FIELD

The present invention involves treating medical conditions involving dysfunctional cells, such as cancer cells, cells having proteostasis dysfunction, and cells with metabolic dysfunction, and more specifically to systems and methods for selectively modulating endoplasmic reticulum stress, proteostasis, and metabolic adaptation in cells associated with medical conditions where such pathways are dysregulated. In some embodiments, the invention involves devices and methods for applying an alternating magnetic field having defined characteristics to a target area of a patient's body. Systems of the present invention may be implanted, or worn by the patient exterior to the body.

### BACKGROUND OF THE INVENTION

Many medical conditions, such as cancer or Type II diabetes, involve dysfunctional cells with abnormal metabolic functions, which result in internal stress within the cell. Cancer cells involve rapid and uncontrolled proliferation, which can result in cellular stress as the cancer cell engages in functions such as protein synthesis and folding, energy regulation, reproduction, and other functions. In many other diseases, including metabolic disorders, autoimmune disorders, and neurodegenerative diseases, cellular stress is also high.

While pharmacological, radiological, and surgical therapies are available in many instances, there is a need for additional therapies to modulate cellular stress in dysfunctional cells. One cellular structure that plays a crucial role in key cellular processes is the endoplasmic reticulum (ER), which is a network of membrane-bound sacs and tubes found in eukaryotic cells. The ER is involved in protein synthesis and folding, lipid synthesis, calcium storage, removing cellular toxins, and in transporting molecules to other cellular or extracellular locations.

The ER includes two distinct components known as the rough ER and the smooth ER, respectively. Both the rough and smooth ER are part of a single, continuous membrane system extending from the nucleus and throughout the cytoplasm. The internal space of the ER is referred to as the lumen, and is where proteins and lipids are processed and modified. The ER consists of flattened sacs called cisternae and tubular structures, the relative portions of which vary according to cell type and function.

Protein synthesis is performed in the rough ER, which includes ribosomes attached to the rough ER surface that give it a rough appearance under a microscope. Ribosomes synthesize proteins by translating messenger RNA (mRNA) into amino acid changes, which are then directed into the lumen of the rough ER, where protein folding and additional modifications (e.g., glycosylation) are performed. The rough ER also performs a quality-control function known as the ER-associated degradation (ERAD), which identifies misfolded proteins for degradation.

Lipid synthesis is performed in the smooth ER, which does not include ribosomes. The smooth ER also removes harmful substances from the cell by modifying them into forms that can be excreted, and stores calcium ions and releases them to regulate certain cellular processes. In some cells, the smooth ER is involved in glycogen breakdown and glucose metabolism. Finally, the smooth ER stores and regulates the release of calcium into the cytoplasm to regulate various cellular processes.

In some embodiments, the present disclosure involves systems and methods to regulate ER stress, including the maintenance of protein homeostasis (i.e., proteostasis), and modulating the unfolded protein response (UPR), which occurs when the ER experiences stress because of an accumulation of misfolded proteins. The UPR is a signaling pathway that increases the production of chaperones to increase the protein-folding capacity of the ER, reducing protein synthesis to alleviate the accumulation of unfolded proteins, and performing ER-associated degradation (ERAD) by identifying and removing misfolded or damaged proteins to maintain proteostasis.

Cancer cells inherently have higher ER stress and proteostasis burden, as well as significantly elevated metabolic activity, rendering them susceptible to therapies that modulate (typically by further increasing) ER stress. Similarly, in many neurodegenerative diseases, many neuronal cells exhibit chronically high ER stress. Metabolic and autoimmune disorders likewise involve cells with high ER stress, leading to chronic immune system activation and inflammation.

Cancer cells exist in a highly stressed microenvironment characterized by hypoxia (low oxygen availability), nutrient deprivation, and oncogene-driven hyperproliferation. Their poor vascularization forces tumors to operate under constant oxidative stress, leading to low oxygen availability. Rapid tumor expansion leads to competition for glucose, amino acids, and lipids to maintain growth rates, and the uncontrolled cell division forces excessive protein synthesis, overloading the ER. Accordingly, ER stress in cancer cells is inherently higher than in non-cancer cells because they are under continuous pressure to synthesize and fold substantial amounts of proteins to sustain their uncontrolled growth. In addition, they rely on the UPR to survive ER stress, in contrast to non-cancer cells which often undergo apoptosis (i.e., self-programmed death) when ER stress exceeds tolerable levels. Finally, they exhibit increased proteasome activity to degrade misfolded proteins, which are exploited by proteasome inhibitors (e.g., Bortezomib, Carfilzomib, Ixazomib) in some pharmacological cancer treatments.

In addition to radiation and chemotherapeutic agents, other therapies involving different modes of action have been used to treat tumor cells, including without limitation ultrasonic and electrical therapies. Electrical currents and electrical fields have been used for decades for medical purposes. In one type of electrical therapy, insulated electrodes have been used to treat cancer cells and other rapidly proliferating cells by applying AC electric fields at frequencies of 50-500 KHz and electric field strengths of about 10-1000 V/m to a target body area that includes such cells. Such therapy is often referred to as TC (“tumor curing”) field or TTF (“tumor treatment field”) therapy.

In U.S. Pat. No. 6,868,289, which is hereby incorporated by reference in its entirety, a method and apparatus are disclosed for destroying rapidly proliferating cells using insulated electrodes to generate an electric field. In U.S. Pat. No. 8,019,414, also hereby incorporated by reference in its entirety, a method of killing or destroying cancer cells is disclosed that involves applying an electric field together with another cancer therapy such as radiation or chemotherapy drugs. However, the use of electric fields to destroy cancer cells, while effective at certain frequencies and electrical field strengths, is limited in many practical respects including the requirement of intimate contact with tissue (e.g., skin) of the patient at all times during the treatment. This may require shaving all hair from the skin. In addition, the electrodes frequently cause skin irritation at the electrode contact site. For example, in one recent study of TTF therapy, forty-three percent (43%) of patients experienced some skin irritation, with 1% reporting severe skin irritation. The relatively high incidence of skin irritation or pain may prohibit the therapy in sensitive body areas (e.g., breast tissue, etc.). TTF therapy also involves the use of high voltages. For this reason, patients must be careful in performing everyday activities having a risk of water exposure (e.g., showering, exercise (sweating), or even exposure to rain).

The use of electrodes in direct contact with the patient's skin presents a risk of burning or heating of tissue adjacent to the electrodes. Because of this risk (and buildup of dirt, oils, etc.), the electrodes in TTF therapy systems typically require frequent replacement (e.g., twice each week). Patients wearing TTF electrodes on the scalp reported headaches related to wearing the electrodes 24 hours a day.

TTF electrodes must also be placed by trained users (e.g., technicians or physicians). Because the treatment is highly localized (i.e., between the electrodes), precise location of the cancer/tumor must first be performed, and the electrodes must be placed with a high degree of accuracy to create an electric field that passes through it. If the electrodes are slightly off of optimal placement, the treatment may result in suboptimal results.

In addition, the power requirements (e.g., high voltages) for generating appropriate electric fields (e.g., at least 10 V/m) result in bulky and/or heavy electronics boxes that must be coupled to the electrodes. One clinical study showed a high rate of falls in patients carrying these cumbersome TTF electronics boxes.

There is a need for safer therapies that may be applied for longer durations to destroy cancer or other rapidly dividing cells, and for modulation of cellular stress responses in non-cancer conditions involving elevated levels of cellular stress. In addition, wearable and non-bulky systems are needed to permit ambulatory, long duration treatments. Finally, there is a need for therapy systems that do not require trained patients or clinicians for setup.

### SUMMARY

In one embodiment, the invention comprises a system for treating dysfunctional cells in a target body area of a patient by modulating cellular stress in the dysfunctional cells, comprising: a) an alternating polarity (AP) magnetic field generator; b) one or more AP electromagnetic coils coupled to the AP magnetic field generator and adapted to be coupled to the target body area, wherein the one or more AP electromagnetic coils are adapted to be energized by an electrical signal from the AP magnetic field generator to generate and apply to the target body area an AP magnetic field having a first frequency within a range of 100 Hz-1 MHz, a field strength within a range of 0.1-10 mT; and c) a controller to control the AP magnetic field generator, the one or more AP electromagnetic coils, and at least one of the first frequency and the field strength; wherein the AP magnetic field is adapted to modulate cellular stress in the dysfunctional cells by at least one of: 1) inducing the formation of micropores in the cell membrane of at least a portion of the dysfunctional cells; 2) inducing thinned areas in the glycocalyx of at least a portion of the dysfunctional cells; 3) inducing the release of one or more of cytokines and immune-modulating molecules from at least a portion of the dysfunctional cells; 4) inducing stress in the endoplasmic reticulum (ER) of at least a portion of the dysfunctional cells; 5) inducing apoptosis in at least a portion of the dysfunctional cells; 6) inducing dormancy in at least a portion of the dysfunctional cells; 7) inducing a change in phenotype of the dysfunctional cells to a less harmful phenotype; and 8) inducing the infiltration of cytotoxic substances from the TME at least a portion of the dysfunctional cells.

In one embodiment, the invention comprises a system for treating cancer cells in a target body area of a patient by modulating at least one of ER stress, proteostasis, metabolic homeostasis, oxidative stress, mitochondrial dysfunction, and immunogenic cell death (ICD), comprising: a) an alternating polarity (AP) magnetic field generator; b) one or more AP electromagnetic coils coupled to the AP magnetic field generator and adapted to be coupled to the target body area, wherein the one or more AP electromagnetic coils are adapted to be energized by an electrical signal from the AP magnetic field generator to generate and apply an AP magnetic field having a frequency of 0.01 Hz-1 MHz and a field strength of 0.1 mT-10 mT, wherein the waveform of the AP magnetic field comprises at least one of a static, oscillating, and a dynamically modulated waveform; and c) a controller to control the AP magnetic field generator, the one or more AP electromagnetic coils, and at least one of the first frequency and the field strength; wherein the AP magnetic field is adapted to induce cellular stress responses by at least one of: 1) inducing oxidative stress and disrupting redox homeostasis; 2) causing mitochondrial membrane depolarization, leading to metabolic dysfunction and apoptosis sensitization; 3) dysregulating proteostasis by impairing protein degradation, proteasome activity, or autophagy, leading to intracellular accumulation of misfolded proteins; 4) interfering with global or selective protein translation via ribosomal function modulation; and 5) triggering metabolic adaptation responses, including glucose and glutamine dependency shifts in tumor cells.

In another embodiment, the invention comprises a system for modulating at least one of: endoplasmic reticulum (ER) stress, proteostasis, or cellular homeostasis in neurodegenerative diseases, comprising: an alternating polarity (AP) magnetic field generator; one or more AP electromagnetic coils coupled to the AP magnetic field generator and adapted to be coupled to a target body area, wherein the one or more AP electromagnetic coils are adapted to be energized by an electrical signal from the AP magnetic field generator to generate and apply an AP magnetic field having a frequency of 0.01 Hz-1 MHz and a field strength of 0.1 mT-10 mT, including static, oscillating, and dynamically modulated waveforms; a controller to control the AP magnetic field generator, the one or more AP electromagnetic coils, and at least one of the frequency or field strength; wherein the AP magnetic field is adapted to prevent or mitigate neurodegeneration by at least one of the following: enhancing adaptive ER stress responses to mitigate proteotoxic stress and support neuronal resilience; inducing controlled ER stress preconditioning to improve neuronal resistance to future insults; reducing intracellular accumulation of misfolded proteins by increasing ER-associated degradation (ERAD) and proteasomal function; restoring ER calcium homeostasis to prevent ER stress-induced neuronal apoptosis; modulating mitochondrial function and oxidative stress to support neuronal metabolic homeostasis; regulating glial cell activation and neuroinflammation to prevent excessive immune-mediated neurotoxicity; and promoting synaptic plasticity and neuronal resilience against oxidative and metabolic stress.

In a further embodiment, the invention comprises a system for modulating at least one of: endoplasmic reticulum (ER) stress, immune homeostasis, or metabolic adaptation in metabolic and autoimmune disorders, comprising: an alternating polarity (AP) magnetic field generator; one or more AP electromagnetic coils coupled to the AP magnetic field generator and adapted to be coupled to a target body area, wherein the one or more AP electromagnetic coils are adapted to be energized by an electrical signal from the AP magnetic field generator to generate and apply an AP magnetic field having a frequency of 0.01 Hz-1 MHz and a field strength of 0.1 mT-10 mT, including static, oscillating, and dynamically modulated waveforms; and a controller to control the AP magnetic field generator, the one or more AP electromagnetic coils, and at least one of the frequency or field strength; wherein the AP magnetic field is adapted to restore ER stress balance and cellular homeostasis by at least one of the following: modulating ER stress homeostasis in pancreatic B-cells to prevent stress-induced apoptosis and enhance adaptive ER stress preconditioning for improved insulin secretion capacity; reducing systemic inflammation by modulating ER stress responses in immune cells; enhancing lipid metabolism, mitochondrial function, and metabolic flexibility in metabolic tissues to support energy homeostasis; suppressing chronic ER stress-induced cytokine storms in autoimmune disorders; modulating gut barrier function and microbiome-associated inflammation to regulate immune activation; and restoring proteostasis balance in immune cells by regulating ER-associated degradation (ERAD), autophagy, and protein chaperone systems to prevent autoimmunity-related ER stress dysfunction.

### BRIEF DESCRIPTION OF THE DRAWINGS

FIG. 1 is a diagram illustrating a mechanism of action of magnetic field therapy (MFT) providing an alternating polarity (AP) magnetic field to a target body area of a patient's body.

FIG. 2 is a flow chart illustrating a cascade of events caused by MFT when applied to dysfunctional cells such as cancers, according an embodiment of the invention.

FIGS. 3A and 3B shows an experimental protocol (FIG. 3A) and results (FIG. 3B) of an experiment investigating the effect of MFT on cell membrane permeability, according to one embodiment of the invention.

FIG. 4 is a graph of flow cytometry results indicating that MFT increases ER stress in cancer cells, according to one embodiment of the invention.

FIGS. 5A and 5B illustrate the effect of MFT on the cell reproduction cycle of human triple negative breast cancer (TNBC) cells, according to one embodiment of the invention.

FIGS. 6A and 6B show the synergistic effects of MFT with metformin (FIG. 6A) and homoharringtonine (HHT) (FIG. 6B) in increasing metabolic and ER stress in TNBC cells, according to one embodiment.

FIG. 7 is a Principal Component Analysis (PCA) graph of bulk RNA sequencing of TNBC cells, showing increased transcriptional changes induced by MFT over time, according to one embodiment.

FIGS. 8A and 8B show results of a gene set enrichment analysis (GSEA) on bulk-RNA sequencing of MDA-MB-231 TNBC cells.

FIGS. 9A-9C show the results of a global proteomics data analysis, including a list of upregulated proteins (FIG. 9A), downregulated proteins (FIG. 9B), and a Gene Ontology (GO) enrichment analysis of MDA-MB-231 TNBC cells (FIG. 9C.

FIG. 10 is a block diagram of a system for providing an alternating polarity (AP) magnetic field to a target body area of a patient's body, according to one embodiment for selectively treating dysfunctional cells.

FIGS. 11A-11C illustrate improved survival in human patients having high neutrophil gene expression for a group of genes expressed during MFT in animals.

### DESCRIPTION

Exemplary embodiments of the present disclosure are illustrated in the drawings, which are illustrative rather than restrictive. No limitation on the scope of the technology or on the claims that follow is to be implied or inferred from the examples shown in the drawings and discussed here.

The present disclosure involves systems and method of modulating cellular stress responses with a device-based therapy involving the application of alternating-polarity magnetic fields to modulate certain cellular processes in dysfunctional cells. In various embodiments, the present disclosure provides systems and methods for modulating cellular stress responses in dysfunctional cells associated with cancer, neurogenerative diseases such as Alzheimer's disease, Parkinson's disease, and ALS, metabolic disorders such as Type 2 diabetes (T2D), and autoimmune disorders such as lupus and rheumatoid arthritis.

In some embodiments, the invention provides apparatus and methods for treating cells of a patient in a target body area using alternating polarity (AP) magnetic fields at specified frequencies to modulate the ER stress and/or proteostasis burden of cells having elevated levels of ER stress and/or proteostasis burdens. As used herein, the terms “magnetic field therapy” and “MFT” refer to systems and methods for applying AP magnetic fields at specified frequencies and magnetic field strengths to dysfunctional cells to modulate cellular stress responses.

Without being bound by theory, it is believed that MFT induces stress in the endoplasmic reticulum (ER) of cancer cells by two primary mechanisms: interaction with glycans and the formation of selective micropores in cell membranes, and disruption of ion homeostasis to increase ER stress and proteostasis imbalance. Cancer cells have an altered glycocalyx composition compared to normal cells. The glycocalyx is a carbohydrate-rich layer covering the outer surface of the plasma membrane of a cell, and consists of negatively charged glycoproteins, glycolipids, and proteoglycans. These oligosaccharides are attached to proteins and lipids, and the glycocalyx forms a protective barrier shielding the cell from mechanical and chemical damage. Additional details on certain aspects of MFT may be found in the following US patents, each of which is hereby incorporated by reference in its entirety: U.S. Pat. Nos. 11,027,143; 11,344,739; 11,344,740; 11,577,089; 11,583,692; 11,391,593; and 11,944,837.

The altered electrical charge profile of cancer cells makes them more electrically active and responsive to Magnetic Field Therapy (MFT). More specifically, cancer cells exhibit a higher density of sialylated and sulfated glycans, making their surface significantly more negatively charged than normal cells. Without being bound by theory, MFT selectively interacts with these glycans, generating high localized magnetic fields at the plasma membrane. This interaction weakens lipid bilayer integrity, forming micropores in the cell membrane through a process similar to electroporation, but without permanent membrane damage. These micropores disrupt ionic balance and mechanical stability, triggering ER stress as the cell attempts to restore homeostasis. Because cancer cells already have elevated ER stress and dysregulated ion homeostasis, making them less capable of buffering further ionic stress. The additional stress added by MFT may push them beyond survival thresholds. MFT further disrupts the already-stressed ion balance in cancer cells because the micropores result in uncontrolled ion leakage into the cytoplasm, depleting the ER of calcium (Ca2+) ions and increasing ER stress. Membrane repair responses of the cell overload proteostasis efforts, further increasing ER stress. In addition, the enhanced cell membrane permeability caused by the micropores may increase the susceptibility of the cancer cell to immune recognition by migration of intracellular components (such as proteins) outside the cell into the tumor microenvironment.

The ER depends on tightly regulated ion concentrations, particularly calcium (Ca²⁺), potassium (K⁺), and chloride (Cl⁻), to maintain proteostasis and signaling integrity. Without being bound by theory, parameters of Magnetic Field Therapy (MFT) can be selected to alter intracellular ion homeostasis, indirectly inducing ER stress through a variety of mechanisms. First, MFT exposure perturbs plasma and ER membrane ion channels (e.g., SERCA, ORAI, ClCN2) responsible for regulating ion flux. This causes a rapid efflux of Ca²⁺ from the ER into the cytosol, depleting ER calcium stores required for protein folding and chaperone function. This depletion impairs protein folding and activates the Unfolded Protein Response (UPR). Increased cytosol Ca²⁺ levels triggers oxidative stress and mitochondrial dysfunction. In addition, Cl⁻ channel (ClCN2) inhibition by MFT also disrupts ER ionic homeostasis, further increasing ER stress. Further, changes in K⁺ homeostasis affect mitochondrial-ER communication, amplifying proteotoxic stress. The loss of ionic balance forces the ER to upregulate stress signaling pathways (PERK, ATF4, CHOP), pushing cancer cells toward apoptosis or dormancy.

MFT parameters can be selected to exploit inherent vulnerabilities in cancer cells, including their highly altered glycocalyx composition, high ER stress burden, proteostasis dependence, and metabolic adaptations. In some cases, MFT may cause stress-induced apoptosis and cell death. This occurs when MFT causes the cancer cell to exceed its stress tolerance and activate pro-apoptotic pathways via ATF4-CHOP signaling, triggering cell death and tumor shrinkage in ER stress-sensitive cancer types.

However, not all tumor cells immediately undergo apoptosis upon therapy-induced ER stress. Instead, MFT can result in several alternative outcomes that still contribute to tumor suppression. These include induced dormancy, increased inflammatory signaling, disruption of cancer cell differentiation and reversal of malignant phenotype, and ER stress-induced metabolic vulnerability. Some cancer cells under persistent ER stress enter a dormant state (quiescence) rather than undergoing apoptosis. This occurs through PERK-eIF2α signaling, which reduces global protein translation, thereby slowing tumor growth. Dormant cancer cells are more sensitive to MFT co-therapies, especially those targeting the tumor microenvironment (TME). Dormancy can be used to convert aggressive cancers into slow-growing, therapy sensitive diseases while preventing rapid resistance development.

MFT may cause ER stress-induced pro-inflammatory signaling that activates the patient's immune system for some cancer cells. This can occur through UPR-induced NF-κB activation, which in turn leads to secretion of IL6, CXCL8, S100A8/A9, making tumors more detectable to immune cells. In addition, the release of damage-associated molecular pattern (DAMP) molecules increases recruitment of macrophages, neutrophils, dendritic cells, and cytotoxic T cells to the cancer area. Pro-inflammatory signaling can render cancer cells more susceptible to immunotherapies such as checkpoint inhibitors, adoptive T-cell therapy, macrophage activation therapies.

MFT may also promote phenotype reversal in some cancer cells. The magnetic fields provided in MFT may interfere with cancer stem-like properties, forcing the cells to dedifferentiate and/or re-differentiate. Increased ER stress has been linked to increased cancer cell plasticity, and prolonged stress that is possible using MFT may force some cancer cells into a less aggressive state. It is known that in some cancers, such as glioblastomas and some breast cancers, inhibiting proteostasis rewires cell fate decisions. Changes in phenotype may reduce the therapy-resistant population in a group of cancer cells, slowing tumor progression.

Persistent ER stress caused by MFT may also force metabolic rewiring to enable the cancer cells to sustain ATP demand and manage oxidative stress. MFT disrupts glucose and glutamine metabolism, increasing reliance on external nutrient sources. This makes the cancer cells sensitive to metabolic inhibitors such as metformin and glutaminase inhibitors. The result is weakened cancer cell survival under metabolic stress.

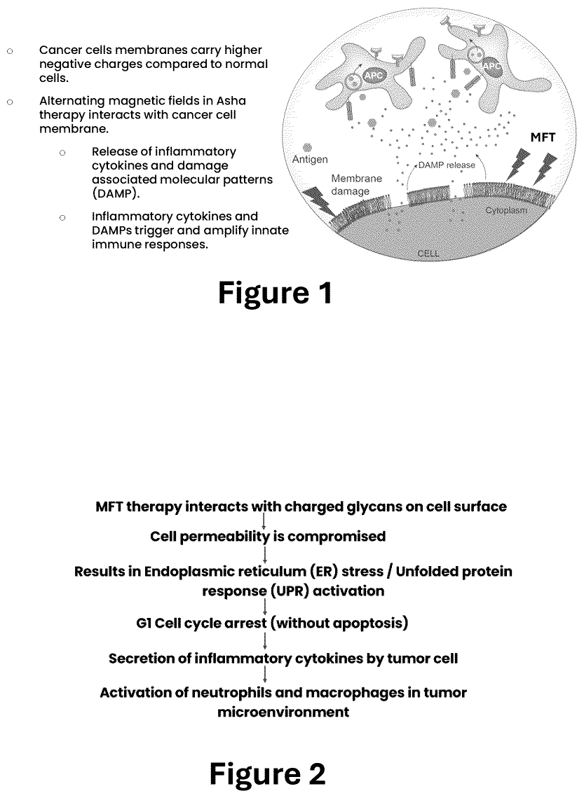

FIG. 1 is a diagram showing the interaction of MFT magnetic fields with the highly negatively charged glycocalyx of cancer cells, and suggests a potential mechanism of action (MoA) for how MFT interacts with the glycocalyx of cancer cells and causes the release of molecules that stimulate the patient's immune system. FIG. 1 proposes that cancer cell membranes, due to their higher negative charge from glycans (glycocalyx), selectively interact with alternating magnetic fields from MFT. This interaction may lead to membrane permeability disruption via micropores and/or thinned areas within the glycocalyx, resulting in the release of inflammatory cytokines and damage-associated molecular patterns (DAMPs) into the tumor microenvironment (TME), with subsequent activation of antigen-presenting cells (APCs) and amplification of innate immune responses against the cancer.

FIG. 2 expands upon the concept of FIG. 1, showing a cascade of events caused by MFT applied to dysfunctional cells such as cancers. The interaction of magnetic fields with glycans compromises membrane integrity, leading to increased ER stress and Unfolded Protein Response (UPR) activation. This may cause G1 cell cycle arrest without apoptosis, followed by inflammatory cytokine secretion by cancer (tumor) cells, driving the activation of neutrophils and macrophages in the TME. Without being bound by theory, FIGS. 1 and 2 align with observed biological effects of MFT, suggesting a novel non-pharmacological strategy for modulating cancer cell biology and enhancing immune-mediated cancer/tumor clearance.

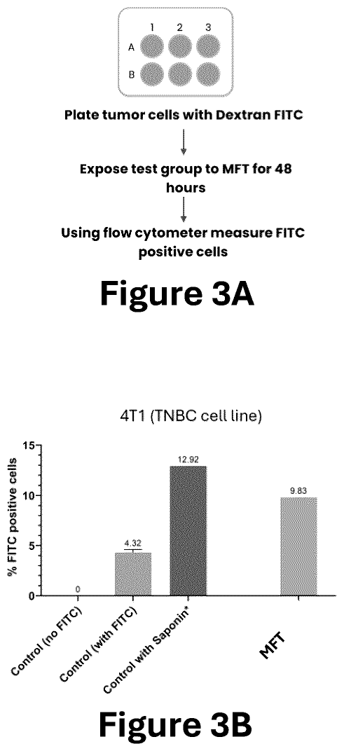

FIGS. 3A and 3B summarize an experiment on the impact of MFT on cell membrane permeability in 4T1 triple-negative breast cancer (TNBC) cells. FIG. 3A shows the experimental procedure: 4T1 TNBC cells were incubated in a 24-well plate (50,000 cells/well) with 5 kDa Dextran-FITC (0.5 mg/ml concentration) and exposed to MFT (50 KHz frequency, 3 mT field strength, 25% duty cycle [250 millisecond on, 750 milliseconds of in a repeating sequence]) for 48 hours. In addition, an untreated control group was not incubated with Dextran-FITC, and a positive control was incubated with the Dextran-FITC and temporarily [HOW LONG?)] exposed to saponin. Flow cytometry analysis using 7-AAD viability dye was then performed to assess FITC uptake in viable cells. FIG. 3B is a graph showing the percentage of FITC-positive cells of untreated control cells and MFT treated cells, demonstrating that MFT treated cells exhibited higher FITC uptake (9.83%) compared to untreated controls (4.32%), though lower than the positive control (saponin-treated, 12.92%), which induces complete membrane permeabilization. These findings suggest that MFT significantly increases cell membrane permeability, and may cause disruptions in ion homeostasis, increased ER stress, and downstream immune activation in cancer cells.

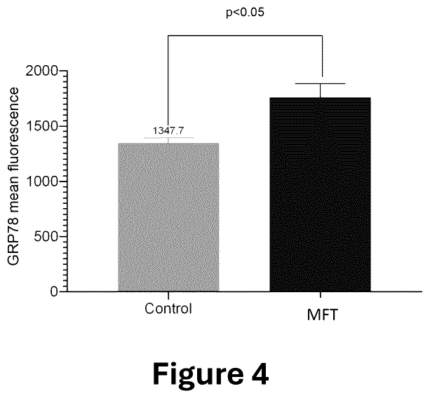

FIG. 4 is a graph of flow cytometry results indicating that MFT increases ER stress in cancer cells. In the experiment, 100,000 MDA-MB-23 human triple negative breast cancer (TNBC) cells were seeded in each well of two 12-well plates. One plate (the test plate) was exposed to MFT (50 KHz frequency, 3 mT field strength, 25% duty cycle [250 millisecond on, 750 milliseconds of in a repeating sequence]) in a modified incubator, while the other (control) plate was maintained in a standard incubator. After 72 hours, cells in both plates were fixed and permeabilized using a BD Cytovix/Cytoperm kit, then stained with a GRP78 antibody. The bar graph shows that cells treated with MFT exhibited significantly higher GRP78 fluorescence intensity compared to untreated controls (p<0.05), suggesting that MFT induces ER stress in cancer cells, potentially making them more susceptible to immune activation or metabolic stress.

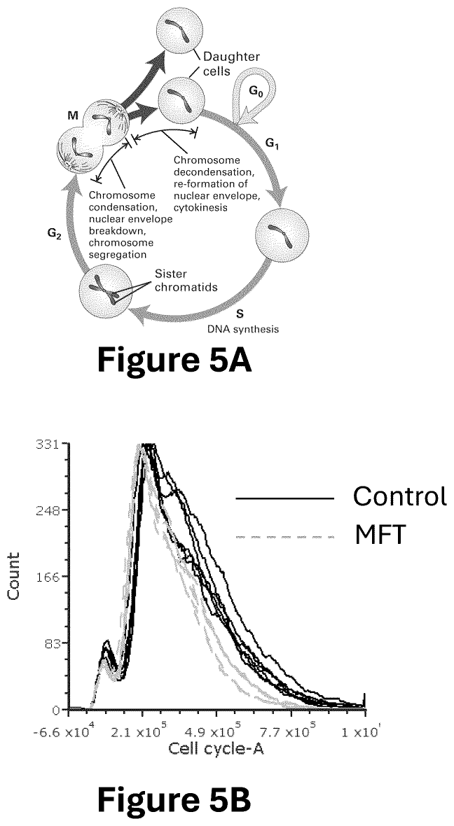

FIGS. 5A and 5B illustrate the effect of MFT on the cell reproduction cycle of MDA-MB-231 human TNBC cells. Similar to the experiment of FIG. 4, 100,000 MDA-MB-231 TNBC cells were seeded in each well of two 12-well plates. A test plate was exposed to MFT (50 KHz frequency, 3 mT field strength, 25% duty cycle [250 millisecond on, 750 milliseconds of in a repeating sequence]) in a modified incubator, while a control plate was maintained in a standard incubator. After 72 hours, cells in both plates were stained with a CellCycle dye, and fluorescence intensity was measured by flow cytometry. FIG. 5A is a reference diagram illustrating the cell reproduction cycle phases. FIG. 5B displays the flow cytometry histograms comparing DNA content in control (black) versus therapy-treated (gray) cells. The shift in the DNA content distribution in therapy-exposed cells suggests a reduction in overall DNA content, indicative of G1 cell cycle arrest or slowed progression through S-phase. This finding aligns with the proposed mechanism of action (MoA) of MFT, wherein ER stress and proteostasis disruption impair cell cycle progression, potentially leading to tumor growth inhibition and increased susceptibility to immune clearance.

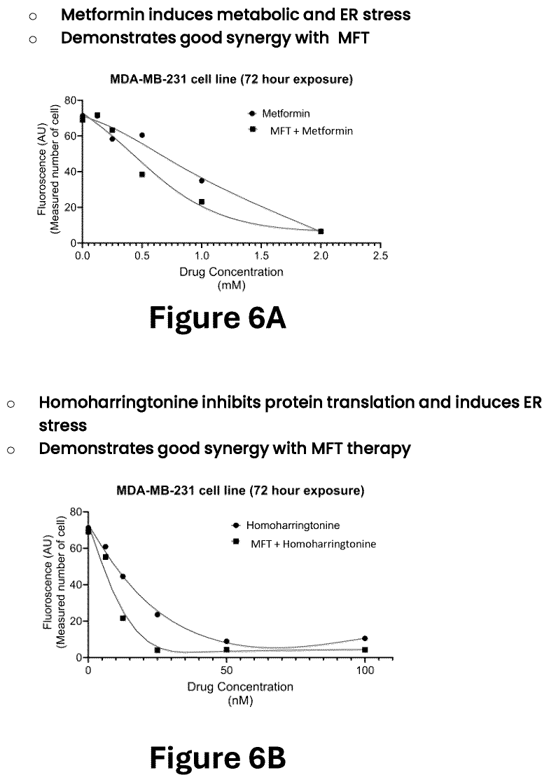

FIGS. 6A and 6B are graphs illustrating the combinatorial effect of MFT either metformin (FIG. 6A) and Homoharringtonine (HHT) (FIG. 6B) on MDA-MB-231 TNBC cells after 72 hours of exposure MFT (50 KHz frequency, 3 mT field strength, 25% duty cycle [250 millisecond on, 750 milliseconds of in a repeating sequence]) as assessed by a Presto Blue proliferation assay. In the experiment, 10,000 cells were placed in each well of two 96 well plates. The test plate was placed in a modified incubator and exposed to MFT, and the control plate (metformin in FIG. 6A; HHT in FIG. 6B) was maintained in a standard incubator and not exposed to MFT. After 24 hours, the culture media was replaced with drug-loaded media (i.e., metformin or HHT), and the plates were returned to their respective incubators for an additional 48 hours of exposure. Upon completion, the media was replaced with Presto Blue-containing media, incubated for 1 hour in a standard incubator, and fluorescence was immediately measured using a plate reader.

FIG. 6A shows that Metformin, known to induce metabolic and ER stress in cancer cells, exhibits enhanced efficacy when combined with MFT (lower line), leading to a greater reduction in cell proliferation. FIG. 6B indicates a similar effect with HHT, a compound known to inhibit protein translation and induce ER stress in cancer cells, where MFT (lower line) further enhances its cytotoxic effects. These findings further suggest that MFT can be used to modulate ER stress, proteostasis, and metabolic homeostasis, thereby making cancer cells more susceptible to additional stress-inducing agents. The observed synergy or additive effects shown in FIG. 6 suggest that MFT not only impairs cancer cell survival by enhancing cellular stress pathways, but also sensitizes cancer cells to additional therapeutic interventions that disrupt ER and/or metabolic function.

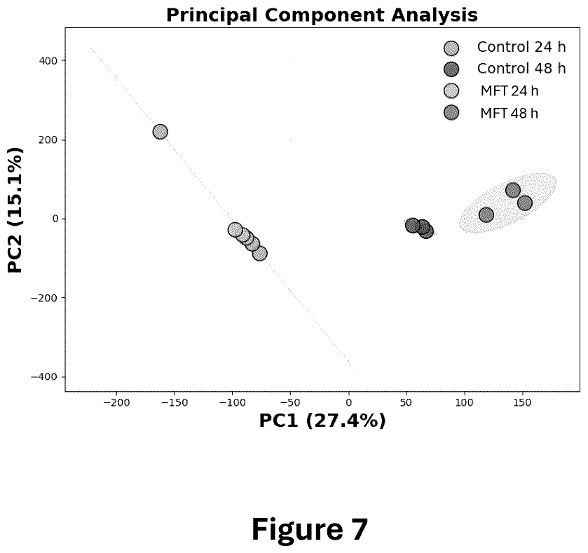

FIG. 7 presences Principal Component Analysis (PCA) of bulk RNA sequencing data from MDA-MB-231 cancer cells exposed to MFT and their respective controls at 24-hour and 48-hour timepoints. A total of 12 samples (6 MFT-exposed, 6 control) were analyzed to assess global transcriptional changes induced by MFT (50 KHz frequency, 3 mT field strength, 25% duty cycle [250 millisecond on, 750 milliseconds of in a repeating sequence]). The PCA plot demonstrates that at 24 hours, there is an emerging divergence between control (c24, large oval in upper left) and therapy-treated (t24, small oval in upper left) groups, suggesting early transcriptional changes induced by the therapy. By 48 hours, this effect becomes more pronounced, as indicated by the greater separation between therapy-treated samples (t48, large oval at right) and control samples (c48, small oval at lower left) along the principal component axes.

The PCA graphs of FIG. 7 indicate that MFT induces progressive transcriptomic alterations over time, leading to distinct clustering of treated vs. control groups. This in turn suggests that MFT elicits a time-dependent cellular response, which may be linked to ER stress, proteostasis modulation, and metabolic adaptation. The increasing divergence at 48 hours suggests that prolonged therapy exposure amplifies these effects, reinforcing its role in modulating cellular pathways relevant to cancer treatment.

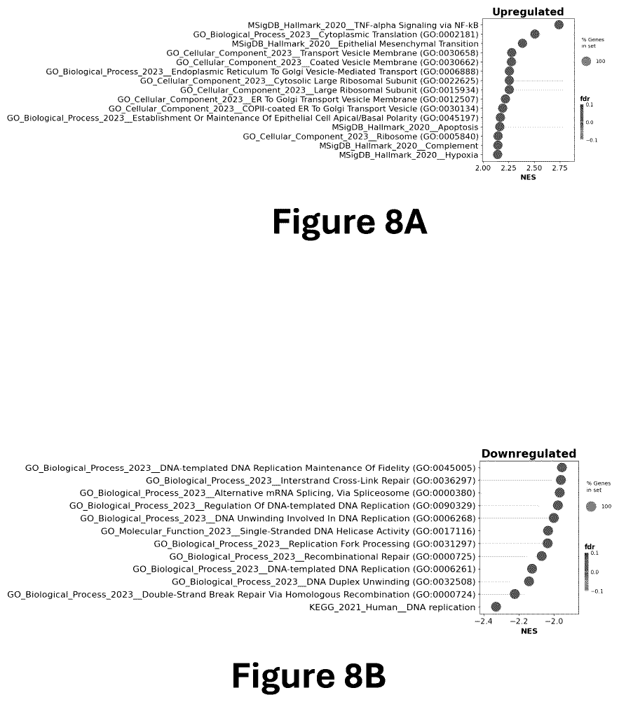

FIGS. 8A and 8B present Gene Set Enrichment Analysis (GSEA) performed on bulk RNA sequencing data from the MDA-MB-231 cells of FIG. 7 that were exposed to MFT for 48 hours, revealing distinct transcriptional alterations. Panel (a) highlights pathways that are significantly upregulated by MFT at 48 hours (FDR<0.05), including TNF-alpha signaling via NF-κB, epithelial-mesenchymal transition (EMT), apoptosis, and ribosomal activity. Figure AA suggests that MFT activates stress-adaptive responses and inflammatory signaling cascades in the cancer cells. Additionally, multiple pathways associated with vesicular trafficking and ER-Golgi transport are upregulated, consistent with enhanced ER stress compensation mechanisms.

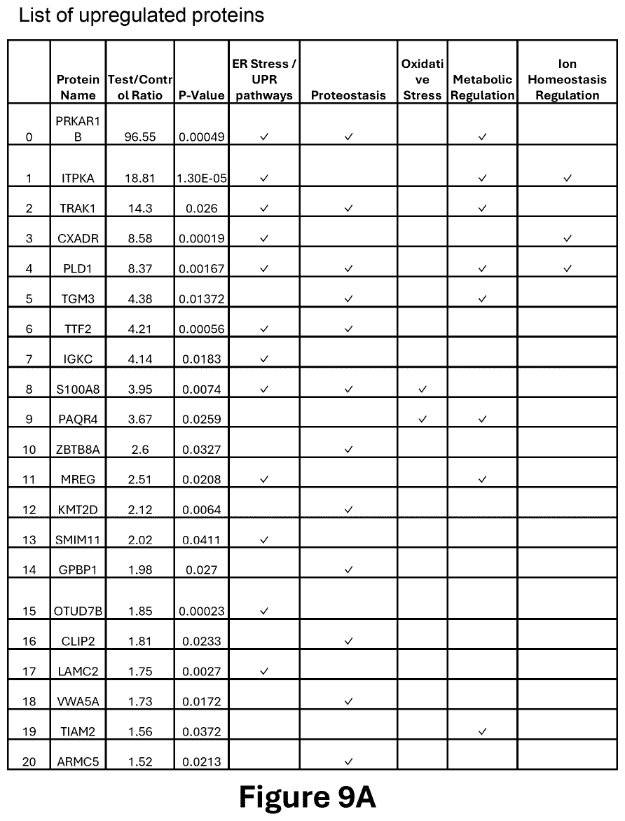

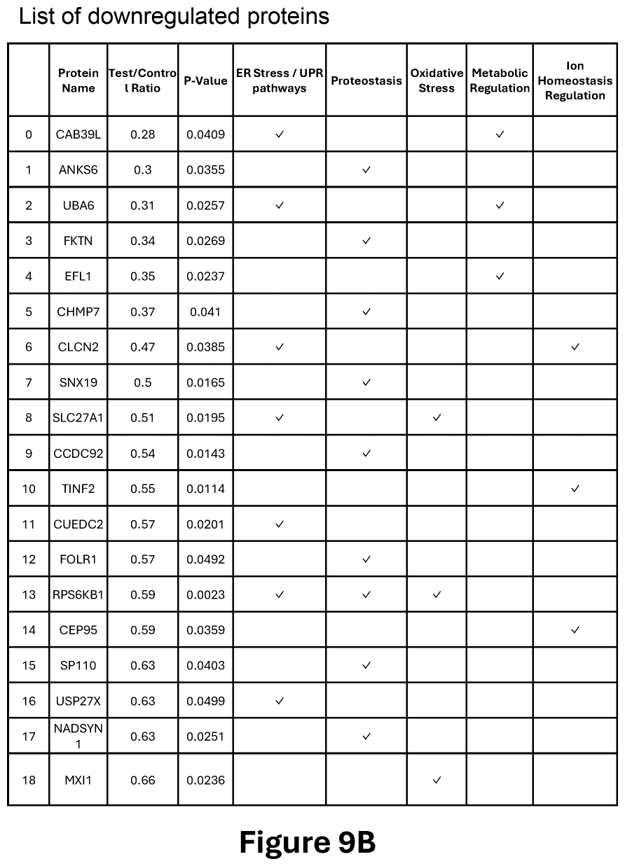

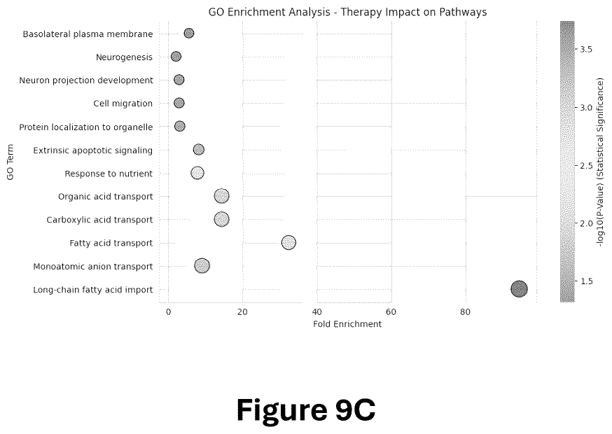

FIG. 9B illustrates pathways that are significantly downregulated by MFT (FDR<0.05). The downregulated pathways involve DNA replication, repair mechanisms (homologous recombination, cross-link repair), and transcriptional regulation, all of which indicate a disrupted proliferative state. The suppression of DNA-templated replication fidelity and recombination repair pathways suggests that MFT interferes with cellular homeostasis, and may induce cell cycle arrest and sensitizing of cancer cells to stress-induced death.

The results shown in FIGS. 7 and 8 further suggest that MFT induces ER stress, disrupts proteostasis, and alters metabolic dependencies, causing transcriptional reprogramming favoring immune system activation and apoptosis while impairing proliferative and survival pathways in cancer cells. The simultaneous activation of immune-modulatory and apoptotic pathways alongside the inhibition of DNA replication and repair further suggests that MFT may be a potent modulator of tumor cell fate.

FIGS. 9A-9C show the results of a global proteomics study conducted on MDA-MB-231 TNBC cells using mass spectrometry, providing an unbiased analysis of protein expression changes following exposure to MFT after 72 hours exposure to MFT (50 KHz frequency, 3 mT field strength, 25% duty cycle [250 millisecond on, 750 milliseconds of in a repeating sequence]). In the study, 300,000 TNBC cells were seeded in each well of two 6-well plates. One plate was placed in a modified incubator and exposed to MFT for 72 hours, and the other plate was placed in a standard incubator for the same time period. The global proteomic analysis was then conducted on the cells using mass spectrometry.

The study included three therapy-treated samples and three control samples, with differential expression analysis identifying 20 significantly upregulated proteins (p_{adj}<0.05, test/control fold ratio>1.5), as shown in FIG. 9A. In addition, the analysis identified 18 significantly downregulated proteins (p_{adj}<0.05, test/control fold ratio<1/1.5), shown in FIG. 9B.

Many of the identified proteins are involved in pathways relevant effects of MFT previously described. For example, upregulated proteins identified are associated with ER stress and the unfolded protein response (UPR), proteostasis, oxidative stress, metabolic regulation, and ion homeostasis. Accordingly, the upregulated proteins illustrate that MFT parameters can be selected to disrupt tumor cell homeostasis. In contrast, several downregulated proteins are associated with cellular processes such as metabolic adaptation, proteostasis, and ion homeostasis, and suggest that MFT disrupts the cancer cells' ability to compensate for therapy-induced stress.

The proteins listed in FIGS. 9A (upregulated proteins) and 9B (downregulated proteins) together show that MFT induces ER stress, disrupts proteostasis, and alters metabolic and ion regulatory pathways, potentially sensitizing cancer cells to immune activation and cell death. The observed protein changes also align with prior transcriptomic and functional assays, further suggesting that MFT broadly impacts cellular stress response pathways in tumor cells.

FIG. 9C depicts the results of a Gene Ontology (GO) enrichment analysis performed on the global proteomic data. The analysis revealed multiple significantly regulated pathways, with fold enrichment values indicating the degree to which these pathways were influenced by MFT. Notably, pathways related to long-chain fatty acid import, monoatomic anion transport, carboxylic acid transport, and response to nutrients were significantly upregulated, suggesting that MFT induces metabolic adaptations in tumor cells. Additionally, extrinsic apoptotic signaling, protein localization, cell migration, and neurogenesis-related pathways were also enriched, further suggesting that MFT can be used to modulate both proteostasis and immune activation. These findings align with prior mechanistic insights suggesting that the therapy disrupts tumor metabolic dependencies, ER stress responses, and immune-stimulatory signaling.

In various embodiments, the present invention may be used to treat one or more cancers such as throat cancer, thyroid cancer, mouth cancer, nose cancer, salivary gland cancer, lung cancer, lung carcinoid tumors, thymic malignancies, tracheal tumors, pancreatic cancer, liver cancer, stomach cancer, kidney cancer, ovarian cancer, prostate cancer, colon cancer and rectal cancer, and blood cancers such as leukemia (e.g., acute myeloid leukemia, chronic lymphocytic leukemia), lymphoma (e.g., Hodgkin's lymphoma, non-Hodgkin's lymphoma), and multiple myeloma (bone marrow-resident plasma cell cancer).

In other embodiments, the present invention may be used to treat one or more neurodegenerative diseases such as Alzheimer's disease (AD), Parkinson's disease (PD), amyotrophic lateral sclerosis (ALS), multiple sclerosis (MS), Huntington's disease (HD), frontotemporal dementia (FTD), and prion-associated diseases.

In still further embodiments, the present invention may be used to treat one or more metabolic diseases such as Type 2 diabetes (T2D), obesity-related metabolic syndrome, non-alcoholic fatty liver disease (NAFLD).

In still further embodiments, the present invention may be used to treat one or more autoimmune diseases such as lupus (SLE), rheumatoid arthritis (RA), inflammatory bowel disease (IBD), Hashimoto's thyroiditis, and psoriasis.

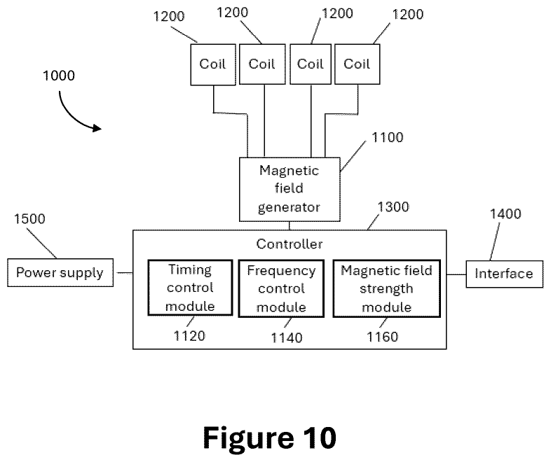

FIG. 10 is a schematic diagram illustrating components of an MFT system 1000. The system includes an alternating polarity magnetic field generator (APMFG) 1100, which generates an electrical signal to energize one or more alternating polarity (AP) electromagnetic coils 1200. These coils produce an AP magnetic field with specific frequency and field strength characteristics. The coils 1200 may vary in size and shape to engage target body areas (e.g., torso, breast, head, neck, throat) for treating dysfunctional cells such as cancer cells, autoimmune-compromised cells, and metabolically dysfunctional cells. A controller 1300 regulates the magnetic fields and overall system operation. A user interface 1400 allows input of treatment parameters and displays system status. A power supply 1500, which may be a battery or AC power source, provides energy and includes circuitry to regulate power for system components.

Controller 1300 includes circuitry (e.g., microcontrollers, memory, firmware) to manage APMFG 1100, AP electromagnetic coils 1200, and interface 1400. FIG. 10 illustrates an embodiment where the AP electromagnetic coils 1200 are energized directly by APMFG 1100. Alternatively, controller 1300 may communicate directly with coils 1200 to enable or disable their operation.

A timing control module 1120 governs the timing of MFT delivered by coils 1200. It can control (e.g., via instructions from a user) therapy durations (e.g., 5 min to 1+ weeks), schedule multiple treatment periods, and adjust field frequency or strength at specified intervals. For instance, it may implement different field parameters during waking and resting periods.

A frequency control module 1140 controls the AP magnetic field frequency, which can range from 100 Hz-1 MHz. It may modify frequency continuously or in steps at programmed rates.

A magnetic field strength control module 1160 controls the AP field strength between 0.1-10 mT, adjusting it through programmed rates or step changes.

Controller 1300 integrates logic, timers, and circuitry for modules 1120, 1140, and 1160. These functions may be combined into submodules or executed by controller 1300 as a whole.

Interface 1400 may include a user input (e.g., keyboard, buttons) for data entry or retrieval. In another embodiment, it may be housed within controller 1300 and use a transceiver to communicate with external devices (e.g., cell phones, tablet computers) for programming and status updates. Alternative designs may omit interface 1400 or integrate it with other system components.

Referring to FIG. 10, APMFG 1100 generates electrical signals to drive coils 1200, producing AP magnetic fields with fixed or variable frequencies. Though depicted with a single wire, each coil 1200 typically connects via a two-wire circuit. In fixed-frequency embodiments, APMFG 1100 generates fields continuously or intermittently based on a duty cycle (e.g., on-time followed by off-time). It can produce various waveforms, including sinusoidal, triangular, and trapezoidal. Some embodiments feature burst mode, applying a predefined number of waveforms at one frequency before switching to another.

AP field frequencies preferably do not exceed 1 MHz, with preferred ranges including 0.1-500 kHz, 0.2-400 kHz, 0.5-300 kHz, 1-200 kHz, 5-150 KHz, 10-100 kHz, and 25-100 KHz. The system may include multiple coils 1200, each operating at different frequencies within these ranges to deliver combined magnetic field effects. For example, one coil may generate a low-frequency field (e.g., 1-5 kHz), while another produces a higher-frequency field (e.g., 50-300 kHz).

Without being bound by theory, AP magnetic fields within 0.5-500 kHz are believed to affect different cellular structures or processes in dysfunctional cells. Each structure or process may respond more effectively to specific frequency sub-ranges, potentially varying by cell type, including different cancer cells, which may require tailored frequencies for modulating cellular stress in dysfunctional cells.

As used herein with respect to one kind of MFT, the term “duty cycle” refers to the fraction of a time period in which an alternating polarity magnetic field is applied to the target body area. The duty cycle may be calculated by the formula

$$duty cycle = \frac{on time}{( on time + off time )}$$

where on time is the period for which the magnetic field is applied to the target body area, and off time is the time following the on time that no magnetic field is applied to the target body area. In one embodiment, the MFT is applied in repeating cycles of on time followed by off time. In a particular example, a 25% duty cycle may involve 250 milliseconds (msec) in which an alternating polarity magnetic field is applied to the target body area, followed by 750 milliseconds in which no magnetic field is applied. It will be appreciated that the same duty cycle can be achieved over different periods, for example 2.5 minutes on time followed by 7.5 minutes off time, or 2.5 hrs on time followed by 7.5 hrs off time.

In alternative embodiments (not shown) to the embodiment of FIG. 10, one or more components of an MFT system, such as coils 1200, magnetic field generator 1100, controller 1300, timing control module 1120, frequency control module 1140, magnetic field strength module 1160, power supply 1500, and interface 1400, may be implantable. In a particular embodiment, coils 1200 may be implanted subcutaneously, while other components such as the field generator 1100, controller 1300, and power supply 1500, may be external, and coupled via wireless connection to supply power, data, and instructions to the implantable coils.

Without being bound by theory, the efficacy of the magnetic field therapy (MFT) is mediated by specific immunomodulatory reprogramming of the tumor microenvironment (TME). Specifically, single-cell transcriptomic analysis (CITE-seq) of murine 4T1 primary tumor identified a distinct population of MFT-induced, interferon-stimulated neutrophils. From these data, a 19-gene ‘Neutrophil Activation Signature’ was derived, comprising genes associated with interferon/antiviral pathways and antigen-processing machinery. To validate clinical utility, this signature was projected onto independent human triple-negative breast cancer (TNBC) datasets (METABRIC, n=338; SCAN-B, n=874). A high neutrophil gene expression score was significantly correlated with reduced mortality and improved overall survival (pooled HR=0.75, p=0.01), identifying said signature as a novel pharmacodynamic biomarker for MFT-induced innate immune activation.

In some embodiments, the invention involves a method for identifying a patient's responsiveness to MFT by determining a neutrophil gene expression (NGE) score derived from one or more of 19 interferon-stimulated genes. In some embodiments, the invention involves monitoring a cancer patient undergoing MFT and/or immune checkpoint blockade, wherein an increase in the NGE score serves as a pharmacodynamic biomarker for therapeutic efficacy and innate immune activation. In some embodiments, the invention involves adjusting MFT parameters including field intensity, frequency, or duty cycle, based on the patient's NGE score to optimize the tumor microenvironment reprogramming. In some embodiments, MFT is administered as a priming strategy for patients receiving non-MFT therapies only when the NGE score indicates an immune-excluded or ‘cold’ tumor state, thereby sensitizing the tumor to subsequent immunotherapy.

A neutrophil gene activation signature was derived from differentially expressed genes in tumor-infiltrating neutrophils treated with alternating polarity (AP) magnetic field therapy (MFT). To determine a neutrophil activation signature, as a first step differentially expressed genes from MFT-treated tumor-infiltrating neutrophils were ranked by wald statistic. The top 100 upregulated genes by Wald statistic (in mouse gene symbols) include: Gm42418, Cmss1, AY036118, Rps28, Ly6e, Rpl13a, Rps29, Rpl37a, Rps23, Filip1I, Tbrg1, Rpl38, Rpl36, Gm16337, Rps26, Rpl26, Gm36043, Rplp1, Rpl13, Rpl27, Rps27, Rps15, Rplp2, Lgals1, Rpl37, Rpl34, Rps21, Rpl24, Spp1, Atp5mpl, 1600014C10Rik, Psme1, Rbm8a, Rps6, Rpl23a, Rps19, Mndal, Gm10076, H2-T22, Rpl11, Sec61g, Rpl31, Manf, Rpl22, Rps14, Rpl17, Rpl39, Atp5d, Uba52, Psmb1, Gm16894, Oasl2, Xaf1, Ifit3b, Ifi2712a, Pkig, Gm4876, Ifit3, Naca, Npc2, Rpl30, Eif2s2, Nf1, Rpl6, Gm37240, Tomm20, Gm20536, Zbp1, Rps15a, Zfas1, Rpl27a, Atp5md, Mif, Mrpl54, Map1b, Psmb8, Rps11, Ndufb9, Rpl19, Mfsd4a, Id3, Rpl15, 2410006H16Rik, Ifit2, Yrdc, 4930453N24Rik, Rpl7, Ifi211, Ndufb1-ps, Uqcrq, Arg1, Cnih4, Rpl18, Rps13, Cstdc4, Ufc1, Atp5e, B2m, Rpl12, Uqcr10.

The foregoing genes encompass multiple gene categories including: canonical interferon-stimulated genes (e.g., Ly6e, Ifit3, Ifit2, Ifi27, Oasl2, Xaf1, Zbp1); antigen-processing machinery (Psmb1, Psmb8, Psme1, B2m); ER stress-associated genes (Sec61g, Tbrg1); neutrophil-associated activation markers (Spp1, Arg1, Mndal); translational and mitochondrial housekeeping transcripts (e.g., ribosomal proteins, Atp5 gene family); and mouse-specific non-coding loci (Gm* genes).

To provide a robust, biologically interpretable neutrophil gene expression (NGE) parameter suitable for application to human bulk RNA-seq data, the following genes were removed from the larger list of genes affected by MFT: (1) ribosomal, mitochondrial, and translation-associated genes representing generic metabolic responses; (2) mouse-specific Gm loci genes lacking human orthologs (e.g., Gm42418, Gm10076, Gm13684)—and Gm16337 was mapped to its closest human antiviral ortholog IFNL3; (3) genes not associated with neutrophil or interferon biology, including RNA-processing factors (e.g., Rbmx, Rbm8a, U2af1, Eif2s2) and endoplasmic reticulum housekeeping genes without known immune relevance; (4) genes whose expression in bulk tumors is dominated by non-neutrophil cell types (e.g., Arg1 in TAMs; Lgals1 in fibroblasts; Cxcl9 in T-cell/endothelial) to avoid confounding.

After removing the foregoing categories of genes, the resulting 19-gene neutrophil expression genes were: LY6E, IFIT3, IFIT2, IFI27, PSMB8, SPP1, HLA-E, OASL, XAF1, ZBP1, PSMB1, PSME1, B2M, TBRG1, FILIP1L, SEC61G, CMSS1, MNDAL, IFNL3. This 19-gene group meets criteria for high statistical induction in MFT-treated neutrophils and has mechanistic relevance to interferon signaling, antigen processing, and neutrophil activation.

The expression status of each of the 18 of the 19 genes in the neutrophil ISG signature was identified for patients in two human triple-negative breast cancer (TNBC) publicly available databases: the METABRIC dataset and the SCAN-B dataset. One gene was missing from each data set: MNDAL gene activation status is absent in the METABRIC microarray, and FILIP1L activation status is absent in SCAN-B RNA-seq. To translate the activation of the neutrophil expression genes into a usable score, a neutrophil activation score based on the activation status of all genes was calculated as the mean normalized expression (z-scores) of available genes.

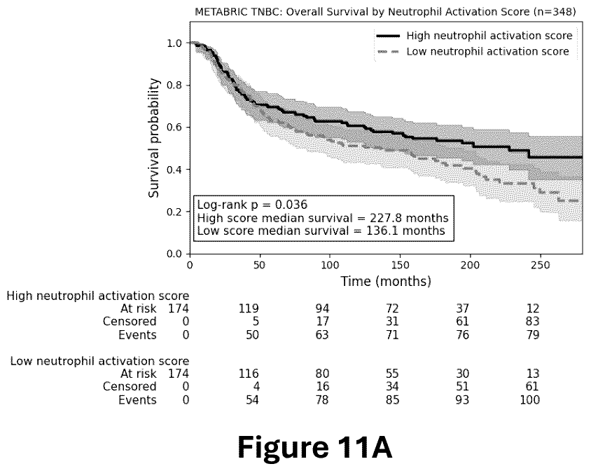

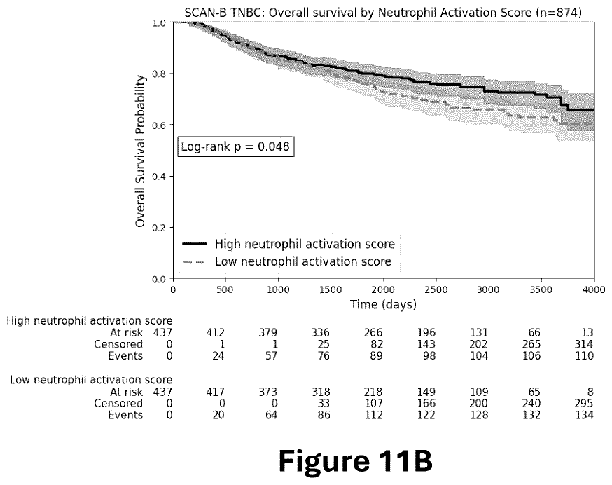

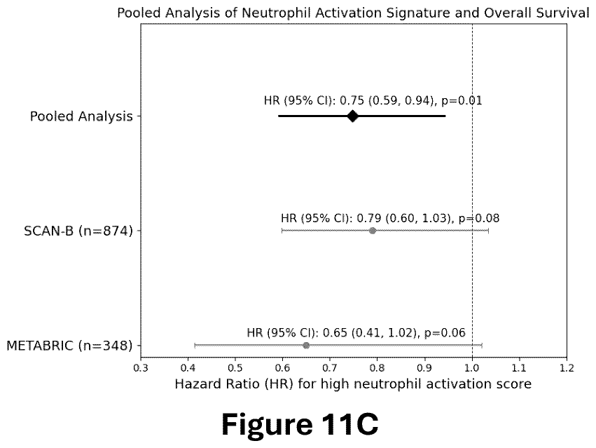

Patients were stratified into high and low groups by median split, and the survival of patients in each group were compared. In METABRIC (n=338) and SCAN-B (n=874), high NGE scores were associated with improved overall survival by Kaplan-Meier analysis (log-rank p=0.036 and p=0.048, respectively; see exemplary Kaplan-Meier curves in FIGS. 11A and 11B). In multivariable Cox models adjusting for nodal status, T stage, tumor size, and stratifying by age, protective effects were observed (METABRIC: HR=0.65, 95% CI 0.41-1.03, p=0.063; SCAN-B: HR=0.79, 95% CI 0.60-1.03, p=0.080). Pooled analysis yields HR=0.75, 95% CI 0.59-0.94, p=0.01 (see exemplary forest plot in FIG. 11C). In various embodiments, the NGE score may be used for: (1) patient selection (e.g., by enrolling patients for MFT with high tumor neutrophil activation score); (2) efficacy monitoring (e.g., measuring NGE score from peripheral blood neutrophils to assess therapy response); or (3) synergistic therapies (e.g., monitoring NGE score during MFT and administering an immunotherapy as a co-therapy when score reaches a co-therapy threshold score.

In one embodiment, the invention comprises a method of treating cancer in a human patient using a neutrophil gene expression (NGE) score of cancer cells, the method comprising: a) obtaining a biological sample of cancer cells comprising neutrophils from the human patient; b) determining an NGE score for the sampled cancer cells based on expression of a plurality of neutrophil-associated genes in the biological sample; and c) performing a further action selected from 1) providing an output indicative of the NGE score; 2) applying MFT to a target body area of the human patient based on the NGE score; 3) selecting at least one parameter of an MFT to be applied to a target body area of the human patient based on the NGE score; 4) providing an indication of efficacy of a therapy received by the patient based on a change in the NGE score from a prior NGE score; and 5) providing an indication of a probability of efficacy of a co-therapy to be administered to the human patient after the determining. In some embodiments, steps (a), (b) and (c) may be repeated one or more times during application of MFT, and a co-therapy may be administered at a time point when the NGE score exceeds an NBE threshold.

In another embodiment, the method may further comprise: d) comparing the NGE score to at least one NGE threshold score; and e) based on the comparing, performing a further action selected from: 1) providing an output indicative of the comparing of the NGE score to the at least one NGE threshold score; 2) applying MFT to a target body area of the human patient based on the comparing; 3) selecting at least one parameter of an MFT to be applied to a target body area of the human patient based on the comparing; 4) providing an indication of efficacy of a therapy received by the patient based on a change in the comparing from a prior comparison; and 5) providing an indication of a probability of efficacy of a co-therapy to be administered to the human patient after the comparing.

In a further embodiment, determining an NGE score may comprise determining an expression of at least a subset of neutrophil-associated genes selected from LY6E, IFIT3, IFIT2, IFI27, PSMB8, SPP1, HLA-E, OASL, XAF1, ZBP1, PSMB1, PSME1, B2M, TBRG1, FILIP1L, SEC61G, CMSS1, MNDAL, and IFNL3.

In another embodiment, determining an NGE score may comprise determining an indication of central tendency of normalized expression values for the plurality of neutrophil-associated genes, wherein the indication of central tendency is selected from a mean, a mode, and an nth percentile value of the normalized expression values of the plurality of neutrophil-associated genes.

In a further embodiment, the method may further comprise: d) changing at least one parameter of a MFT applied to a target body area of the human patient before the determining based on the determining to provide an adjusted MFT; e) applying the adjusted MFT for a first therapy period; f) obtaining an additional biological sample of cancer cells comprising neutrophils from the human patient after the first therapy period; and g) determining an updated NGE score based on the additional biological sample.

In a further embodiment, changing the at least one MFT parameter may comprise changing at least one of a magnetic field frequency, a magnetic field strength, a duty cycle, or a treatment duration. In a further embodiment, the method may further comprise administering a co-therapy selected from immunotherapy, chemotherapy, and radiation therapy, and wherein the co-therapy is initiated in response to the NGE score exceeding an NGE threshold.

In one embodiment, the invention comprises a system for treating cancer in a human patient using a neutrophil gene expression (NGE) score, comprising a non-transitory computer-readable storage medium storing computer-executable instructions that, when executed by a processor, cause the processor to: a) receive, from an RNA sequencer analyzing a biological sample comprising neutrophils obtained from the human patient, gene expression data for a plurality of neutrophil-associated genes; b) determine an NGE score for the plurality of neutrophil-associated genes based on the gene expression data, wherein the NGE score is an indication of central tendency of normalized expression values for the plurality of neutrophil-associated genes, wherein the indication of central tendency is selected from a mean, a mode, and an nth percentile value of the normalized expression values of the plurality of neutrophil-associated genes; and c) perform a further action selected from 1) providing an output indicative of the NGE score; 2) initiating the application of MFT to a target body area of the human patient based on the NGE score; 3) selecting at least one parameter of an MFT to be applied to a target body area of the human patient based on the NGE score; 4) providing an indication of efficacy of a therapy received by the patient based on a change in the NGE score from a prior NGE score; and 5) providing an indication of a probability of efficacy of a co-therapy to be administered to the human patient, based on the NGE score.

In another embodiment, the non-transitory computer-readable storage medium may be further adapted to determine an updated NGE score from an additional biological sample obtained after MFT is applied for a first therapy period, and to provide an output comprising an adjustment of at least one of a frequency, a field strength, a duty cycle, or a treatment duration of MFT based on the updated NGE score.

The particular embodiments disclosed above are illustrative only, as the invention may be modified and practiced in different but equivalent manners apparent to those skilled in the art having the benefit of the teachings herein. Embodiments disclosed and claimed herein may be made and executed without undue experimentation with the benefit of the present disclosure. While the invention has been described in terms of particular embodiments, it will be apparent to those of skill in the art that variations may be applied to systems and apparatus described herein without departing from the concept, spirit and scope of the invention. Examples are all intended to be non-limiting. It is therefore evident that the particular embodiments disclosed above may be altered or modified and all such variations are considered within the scope and spirit of the invention, which are limited only by the scope of the claims.

What is claimed is:

1. A system for treating dysfunctional cells in a target body area of a patient by modulating cellular stress in the dysfunctional cells, comprising:
a) an alternating polarity (AP) magnetic field generator;
b) one or more AP electromagnetic coils coupled to the AP magnetic field generator and adapted to be coupled to the target body area, wherein the one or more AP electromagnetic coils are adapted to be energized by an electrical signal from the AP magnetic field generator to generate and apply to the target body area an AP magnetic field having a first frequency within a range of 100 Hz-1 MHz, a field strength within a range of 0.1-10 mT; and
c) a controller to control the AP magnetic field generator, the one or more AP electromagnetic coils, and at least one of the first frequency and the field strength; wherein the AP magnetic field is adapted to modulate cellular stress in the dysfunctional cells by at least one of:
1) inducing the formation of micropores in the cell membrane of at least a portion of the dysfunctional cells;
2) inducing thinned areas in the glycocalyx of at least a portion of the dysfunctional cells;
3) inducing the release of one or more of cytokines and immune-modulating molecules from at least a portion of the dysfunctional cells;
4) inducing stress in the endoplasmic reticulum (ER) of at least a portion of the dysfunctional cells;
5) inducing apoptosis in at least a portion of the dysfunctional cells;
6) inducing dormancy in at least a portion of the dysfunctional cells;
7) inducing a change in phenotype of the dysfunctional cells to a less harmful phenotype; and
8) inducing the infiltration of cytotoxic substances from the TME at least a portion of the dysfunctional cells.

2. The system of claim 1, wherein inducing stress in the endoplasmic reticulum comprises at least one of:
A) impaired proteostasis;
B) impaired protein degradation;
C) accumulation of at least one of misfolded proteins and unfolded proteins in the ER;
D) impairing proteasome activity;
E) interfering with protein translation;
F) modulating ribosomal function;
G) disrupting calcium ion balance;
H) depletion of calcium ions in the ER;
I) activation of the unfolded protein response (UPR);
J) upregulation of ER chaperones;
K) disruption of the ER-associated degradation (ERAD) function of the ER;
L) overwhelming the protein folding capacity of the ER; and
M) release of damage-associated molecular patterns (DAMPs).

3. The system of claim 1, wherein the AP magnetic field has a first frequency within a range of 0.5-500 kHz and a field strength within a range of 0.2-5 mT.

4. The system of claim 1, wherein the dysfunctional cells comprise at least one of cancer cells, cells associated with a metabolic disorder, cells associated with an autoimmune disorder, and cells associated with a neurodegenerative disease.

5. The system of claim 1, further comprising:
(d) a retaining element for retaining the one or more AP electromagnetic coils in a desired position relative to the target body area during the application of the AP magnetic field to the target body area;
wherein the retaining element is selected from a garment and a bandage, the one or more AP electromagnetic coils, the retaining element, and the controller are each wearable by a user, and the system comprises an ambulatory treatment system capable of treating the dysfunctional cells while the patient is non-stationary.

6. The system of claim 1, wherein the controller comprises a timing control module to perform at least one of:
applying the AP magnetic field continuously to the target body area for a first treatment period;
applying the AP magnetic field intermittently for a second treatment period in alternating on time periods in which the AP magnetic field is applied to the target body area, followed by off time periods in which the AP magnetic field is not applied to the target body area;
applying the AP magnetic field intermittently for one or more circadian treatment periods based on circadian rhythms of the patient; and
applying the AP magnetic field intermittently for one or more third treatment periods at defined times of day.

7. The system of claim 1, wherein the controller comprises a frequency control module to perform at least one of:
applying an AP magnetic field having a single frequency within a range of 1-500 kHz to the target body area;
applying an AP magnetic field having a frequency within a range of 1-500 kHz that varies in a defined pattern.

8. The system of claim 7 wherein the frequency control module further controls the AP magnetic field generator and the one or more AP electromagnetic coils to apply an AP magnetic field having at least one of:
a frequency within a range of 1-500 KHz that varies randomly;
a frequency that varies in a Gaussian distribution in one or more ranges within a range of 1-500 kHz;
a frequency that varies in a non-Gaussian distribution in one or more ranges within a range of 1-500 KHz.

9. The system of claim 1, wherein the one or more AP electromagnetic coils are adapted to generate and apply to the target body area an AP magnetic field adapted to modulate cellular stress in the dysfunctional cells by at least one of:
1) inducing the formation of micropores in the cell membrane of at least a portion of the dysfunctional cells;
2) inducing thinned areas in the glycocalyx of at least a portion of the dysfunctional cells; and
3) inducing stress in the endoplasmic reticulum (ER) of at least a portion of the dysfunctional cells.

10. The system of claim 1, wherein the controller is adapted to control the AP magnetic field generator and the one or more AP electromagnetic coils to generate and apply to the target body area an AP magnetic field having a frequency within a range of 1.0-200 KHz and a field strength within a range of 0.5-2 mT.

11. The system of claim 1, wherein the controller is adapted to control the AP magnetic field generator and the one or more AP electromagnetic coils to generate and apply to the target body area an AP magnetic field comprising a series of pulse bursts, wherein each pulse in each pulse burst comprises a predefined number of AP magnetic waveforms having a frequency within a range of 0.1-500 KHz and a field strength within a range of 0.1-5 mT, and each pulse burst in the series of pulse bursts comprises a series of pulses applied at a pulse frequency within a range of 0.01-1000 Hz and a pulse duration within a range of 0.1-5000 msec.

12. The system of claim 1, wherein the dysfunctional cells comprise at least one of throat cancer cells, thyroid cancer cells, mouth cancer cells, nose cancer cells, salivary gland cancer cells, lung cancer cells, lung carcinoid tumors cells, thymic malignancy cells, tracheal tumor cells, pancreatic cancer cells, liver cancer cells, stomach cancer cells, kidney cancer cells, ovarian cancer cells, prostate cancer cells, colon cancer cells, rectal cancer cells, and blood cancer cells.

13. The system of claim 1, wherein the dysfunctional cells comprise at least one of cells functionally compromised by one or more of Alzheimer's disease (AD), Parkinson's disease (PD), amyotrophic lateral sclerosis (ALS), multiple sclerosis (MS), Huntington's disease (HD), frontotemporal dementia (FTD), prion associated diseases, Type 2 diabetes (T2D), obesity-related metabolic syndrome, non-alcoholic fatty liver disease (NAFLD), lupus (SLE), rheumatoid arthritis (RA), inflammatory bowel disease (IBD), Hashimoto's thyroiditis, and psoriasis.

14. A system for treating cancer cells in a target body area of a patient by modulating at least one of ER stress, proteostasis, metabolic homeostasis, oxidative stress, mitochondrial dysfunction, and immunogenic cell death (ICD), comprising:
a) an alternating polarity (AP) magnetic field generator;
b) one or more AP electromagnetic coils coupled to the AP magnetic field generator and adapted to be coupled to the target body area, wherein the one or more AP electromagnetic coils are adapted to be energized by an electrical signal from the AP magnetic field generator to generate and apply an AP magnetic field having a frequency of 0.01 Hz-1 MHz and a field strength of 0.1 mT-10 mT, wherein the waveform of the AP magnetic field comprises at least one of a static, oscillating, and a dynamically modulated waveform; and
c) a controller to control the AP magnetic field generator, the one or more AP electromagnetic coils, and at least one of the first frequency and the field strength; wherein the AP magnetic field is adapted to induce cellular stress responses by at least one of:
1) inducing oxidative stress and disrupting redox homeostasis;
2) causing mitochondrial membrane depolarization, leading to metabolic dysfunction and apoptosis sensitization;
3) dysregulating proteostasis by impairing protein degradation, proteasome activity, or autophagy, leading to intracellular accumulation of misfolded proteins;
4) interfering with global or selective protein translation via ribosomal function modulation; and
5) triggering metabolic adaptation responses, including glucose and glutamine dependency shifts in tumor cells.

15. The system of claim 14, wherein the AP magnetic field is further adapted to induce at least one of:
6) endoplasmic reticulum (ER) stress leading to unfolded protein accumulation; and
7) intracellular stress affecting protein homeostasis, proteasome dysfunction, or translational dysregulation of protein synthesis.

16. The system of claim 15, wherein the endoplasmic reticulum (ER) stress comprises at least one of disruptions in calcium flux and ion imbalance leading to proteostasis collapse.

17. They system of claim 14, wherein the AP magnetic field induces cellular stress via at least one of:
6) selective micropore formation in tumor cell membranes due to interactions with negatively charged glycans, leading to ion leakage and ER stress activation;
7) disruption of ion homeostasis by altering calcium (Ca²⁺), chloride (Cl⁻), and potassium (K⁺) flux, leading to ER calcium depletion and activation of the Unfolded Protein Response (UPR);
8) inducing sustained activation of the PERK-eIF2α pathway, leading to translational suppression and stress-adaptive signaling;
9) triggering the ATF6 pathway, leading to upregulation of ER chaperones and unfolded protein response (UPR) genes to modulate protein folding capacity;
10) activating the IRE1-XBP1 pathway, increasing ER-associated degradation (ERAD) and secretory pathway adaptation;
11) causing excessive proteotoxic stress, overwhelming ER folding capacity and leading to ERAD failure; and
12) inducing immunogenic cell death (ICD) via at least one of:
a) ER stress-driven exposure of calreticulin;
b) release of damage-associated molecular patterns (DAMPs); and
c) secretion of cytokines and immune-modulatory molecules, including but not limited to ATP, HMGB1, and heat shock proteins (HSPs).

18. The system of claim 14, wherein the AP magnetic field is adapted to at least one of:
6) modulate ER stress in hematopoietic progenitor cells, disrupting proteostasis in malignant cells;
7) induce selective apoptosis and/or dormancy in leukemic or lymphoma cells via ER stress overload;
8) enhance immune activation by increasing infiltration and activation of cytotoxic immune cells, including CD8+ T cells, NK cells, and antigen-presenting cells (APCs), while reducing myeloid-derived suppressor cells (MDSCs) and regulatory T cells (Tregs); and
9) disrupt metabolic dependencies in malignant hematopoietic cells by interfering with at least one of glutamine metabolism, lipid metabolism, glycolysis, oxidative phosphorylation (OXPHOS), and mitochondrial metabolic adaptations required for tumor cell survival.

19. The system of claim 14, further comprising:
d) a retaining element for securing the AP electromagnetic coils to hematopoietic-rich regions, wherein the retaining element comprises at least one of: pants, shorts, thigh wraps, or other wearable garments designed to retain electromagnetic coils over the femur, and a wrap positioned around the pelvic and lumbar region for targeting bone marrow niches.

20. The system of claim 14, wherein at least one of the AP magnetic field generator, the one or more AP electromagnetic coils, and the controller is implantable, and the system is configured to generate and apply an alternating polarity (AP) magnetic field within a localized tumor microenvironment (TME).

21. A system for modulating at least one of: endoplasmic reticulum (ER) stress, proteostasis, or cellular homeostasis in neurodegenerative diseases, comprising:
an alternating polarity (AP) magnetic field generator;
one or more AP electromagnetic coils coupled to the AP magnetic field generator and adapted to be coupled to a target body area, wherein the one or more AP electromagnetic coils are adapted to be energized by an electrical signal from the AP magnetic field generator to generate and apply an AP magnetic field having a frequency of 0.01 Hz-1 MHz and a field strength of 0.1 mT-10 mT, including static, oscillating, and dynamically modulated waveforms;
a controller to control the AP magnetic field generator, the one or more AP electromagnetic coils, and at least one of the frequency or field strength;
wherein the AP magnetic field is adapted to prevent or mitigate neurodegeneration by at least one of the following:
enhancing adaptive ER stress responses to mitigate proteotoxic stress and support neuronal resilience;
inducing controlled ER stress preconditioning to improve neuronal resistance to future insults;
reducing intracellular accumulation of misfolded proteins by increasing ER-associated degradation (ERAD) and proteasomal function;
restoring ER calcium homeostasis to prevent ER stress-induced neuronal apoptosis;
modulating mitochondrial function and oxidative stress to support neuronal metabolic homeostasis;
regulating glial cell activation and neuroinflammation to prevent excessive immune-mediated neurotoxicity; and
promoting synaptic plasticity and neuronal resilience against oxidative and metabolic stress.

22. A system for modulating at least one of: endoplasmic reticulum (ER) stress, immune homeostasis, or metabolic adaptation in metabolic and autoimmune disorders, comprising:
an alternating polarity (AP) magnetic field generator;
one or more AP electromagnetic coils coupled to the AP magnetic field generator and adapted to be coupled to a target body area, wherein the one or more AP electromagnetic coils are adapted to be energized by an electrical signal from the AP magnetic field generator to generate and apply an AP magnetic field having a frequency of 0.01 Hz-1 MHz and a field strength of 0.1 mT-10 mT, including static, oscillating, and dynamically modulated waveforms; and
a controller to control the AP magnetic field generator, the one or more AP electromagnetic coils, and at least one of the frequency or field strength;
wherein the AP magnetic field is adapted to restore ER stress balance and cellular homeostasis by at least one of the following:
modulating ER stress homeostasis in pancreatic β-cells to prevent stress-induced apoptosis and enhance adaptive ER stress preconditioning for improved insulin secretion capacity;
reducing systemic inflammation by modulating ER stress responses in immune cells;
enhancing lipid metabolism, mitochondrial function, and metabolic flexibility in metabolic tissues to support energy homeostasis;
suppressing chronic ER stress-induced cytokine storms in autoimmune disorders;
modulating gut barrier function and microbiome-associated inflammation to regulate immune activation; and
restoring proteostasis balance in immune cells by regulating ER-associated degradation (ERAD), autophagy, and protein chaperone systems to prevent autoimmunity-related ER stress dysfunction.
